(19) United States
(12) Patent Application Publication
(10) Pub. No.: US 2022/0192859 A1
(43) Pub. Date: Jun. 23, 2022

(54) HAND CONTAMINATION PREVENTING DRAINAGE BAG STRUCTURE

(71) Applicant: CHIEN-CHUNG SU, Taichung City (TW)

(72) Inventors: CHIEN-CHUNG SU, Taichung City (TW); HSIN-LIANG HAN, Taichung City (TW)

(21) Appl. No.: 17/559,096

(22) Filed: Dec. 22, 2021

(30) Foreign Application Priority Data
Dec. 22, 2020 (TW) 109145474

(51) Int. Cl. A61F 5/44

(52) U.S. Cl. CPC A61F 5/4405

(57) ABSTRACT

A hand contamination preventing drainage bag structure includes a urine bag and a hand contamination preventing structure disposed on the urine bag. The urine bag has a magnetic member. The hand contamination preventing structure includes a tubular main body. The main body contains a bead for forming a sealed status in the main body and the urine bag. When the present invention is to be used, the magnetic member is moved toward the main body, such that the bead presses the elastic member to form an open status in the main body and the urine bag. Thus, the present invention achieves the convenience of labor-saving, simple, one-handed and fast operation, and also reduces the probability of the hand touching the urine. Therefore, the urine bag improvement structure has greater convenience of usage.

### BACKGROUND OF THE INVENTION

### 1. Field of the Invention

The present invention relates to urine collection bags for collecting urine discharged from a patient through a catheter disposed in the urethra of the patient, and structures for the urine collection hag, more particularly, to a hand contamination preventing drainage bag structure.

### 2. Description of the Related Art

When a patient encounters a major trauma, surgery or illness, the patient usually uses a urine bag. Further, a patient who loses urination function needs to use a catheter directly introduced into the bladder, so that the patient who loses urination function can directly discharge urine into the urine bag. Without such device, the patient lacking urination function is more likely to suffer urethra and bladder infections. Although the conventional method of using urine bags can directly solve the problem of urination of patients, the patient lacking the urination function for a long period has to permanently rely on the usage of urine bag. Also, the incapability of urination causes inconvenience for the daily life of the patient and the family members thereof.

For a common urine collection bag, the inlet tube connected with the catheter placed in the urethra is connected to the upper part of the bag body, and the discharge tube extends from the lower part of the bag body. For example, a conventional urine collection bag has a bag body formed of a transparent or translucent resin sheet, with an inlet tube connected to the upper part of the bag body, a discharge tube connected to the lower part of the bag body, and a suspension appliance fixed to an upper end of the bag body. At the lower end of the bag body, the sheet film is joined into a roughly horizontal line to form a cylindrical penetrating part. Usually, the middle of the discharge tube is sealed, and the front end is inserted into the penetrating part.

When such urine collection bag is used, the catheter placed in the urethra of the patient is connected to the front end of the inlet tube; and when the patient is on the bed, the bag body is suspended on the frame of the bed through a suspending appliance. At this time, because the discharge tube is inserted into the penetrating part, the bed can be prevented from contamination due to contact. In addition, when the urine stored in the bag is discharged, the discharge tube is pulled out from the penetrating part, and the clamp is loosened for removing the urine in the bag body. However, because the discharge tube is flexible and has a certain degree of elasticity, the discharge tube quickly recovers to its original shape from a bent state when being pulled out from the penetrating part, causing the issue of urine splashing from the front end of the tube.

Also, a valve device for a urine collection bag and a urine collection bag having the valve device are developed by the industry. The valve device for the urine collection bag is a valve device installed on the urine discharge part of the urine collection bag. The device comprises a casing for holding the urine discharge tube extending from the bag body, and a sliding part held by the casing, wherein the sliding part is capable of sliding in the transverse direction of the urine discharge tube with respect to the urine discharge tube in the casing. The sliding part moves toward the inner side of the casing when at the forward position, so as to be elastically deformed in the radial direction to be closed, and moves toward the outer side from the casing at the backward position, so as to relieve the elastic deformation of the urine discharge tube to be opened.

The aforementioned invention uses a valve to control the discharge of urine from the urine collection bag. However, a general urine bag is usually replaced after a usage period of approximately two weeks to one month. Due to the repeatedly opening and discharging operations, the gap between the valve switch gate and the tube wall will be enlarged and slippery. As a result, the enlarged gap often causes urine leakage when opening, resulting in possible contamination of user's hand. The improvement upon such issue is desirable.

### SUMMARY OF THE INVENTION

To improve the issues of the conventional urine bag above, the present invention is disclosed after multi-dimensional discussion, improvements, and experiment samples.

The present invention provides a hand contamination preventing drainage bag structure, comprising a urine bag provided with a magnetic member; and a hand contamination preventing structure disposed on the urine bag; the hand contamination preventing structure comprising a T-shaped tubular main body, with a bead and an elastic member disposed in the middle of the main body. By use of the combination of the bead and the elastic member, the main body and the urine bag are in a sealed status. To open the structure, the magnetic member is move toward the T-shaped tubular main body, so that the bead presses the elastic member, whereby the main body and the urine bag are in an open status.

The hand contamination preventing drainage bag structure of the present invention aims at collecting the urine discharged by the patient by magnetic suction, thereby achieving the convenience of labor-saving, simple, one-handed and fast operation, and also reducing the probability of the hand touching the urine. Thus, the urine bag improvement structure has greater convenience of usage.

### BRIEF DESCRIPTION OF THE DRAWINGS

FIG. 1 is a perspective view of the present invention.

FIG. 2 is an exploded view of the present invention.

FIG. 3 is a sectional view of the present invention.

FIG. 4 is a sectional view of the present invention in the sealed usage status.

FIG. 5 is a sectional view of the present invention in the partially sealed usage status.

FIG. 6 is a sectional view of the present invention in the open usage status of magnetic suction operation.

FIG. 7 is a partially sectional view of the present invention in the open usage status of magnetic suction operation.

FIG. 8 is a sectional view in accordance with another embodiment of the present invention in the sealed usage status.

FIG. 9 is a sectional view in accordance with another embodiment of the present invention in the open usage status of magnetic suction operation.

### DETAILED DESCRIPTION OF THE INVENTION

The following content is a further explanation in conjunction with the drawings of the preferred embodiments of the present invention, so as to enable people who are familiar with related technologies of the present invention to implement the embodiments based on the statements in this specification.

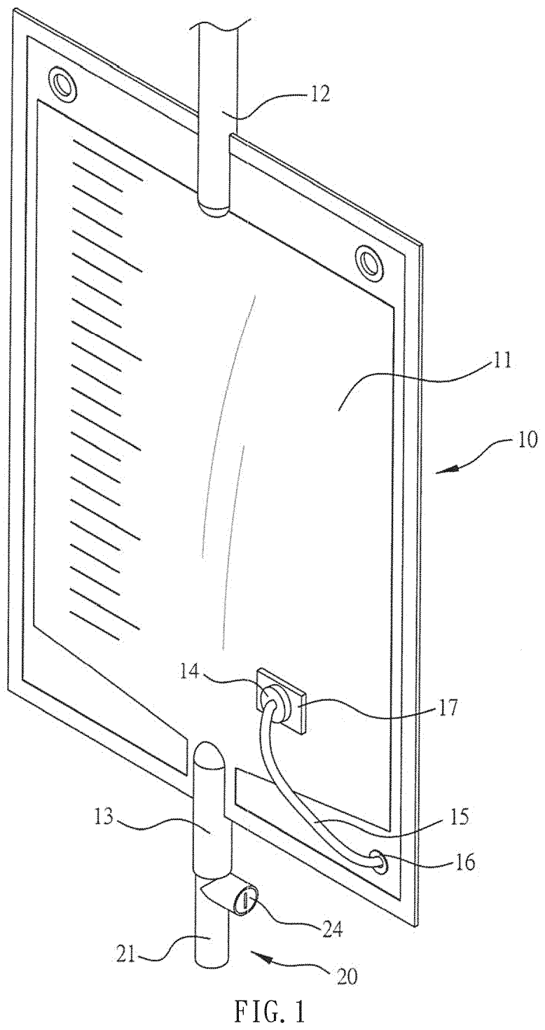

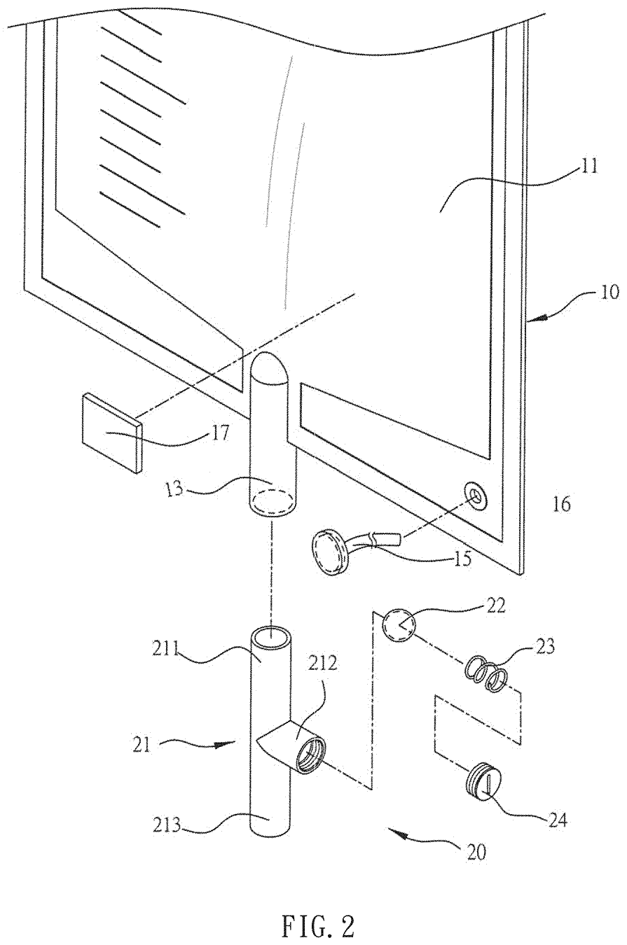

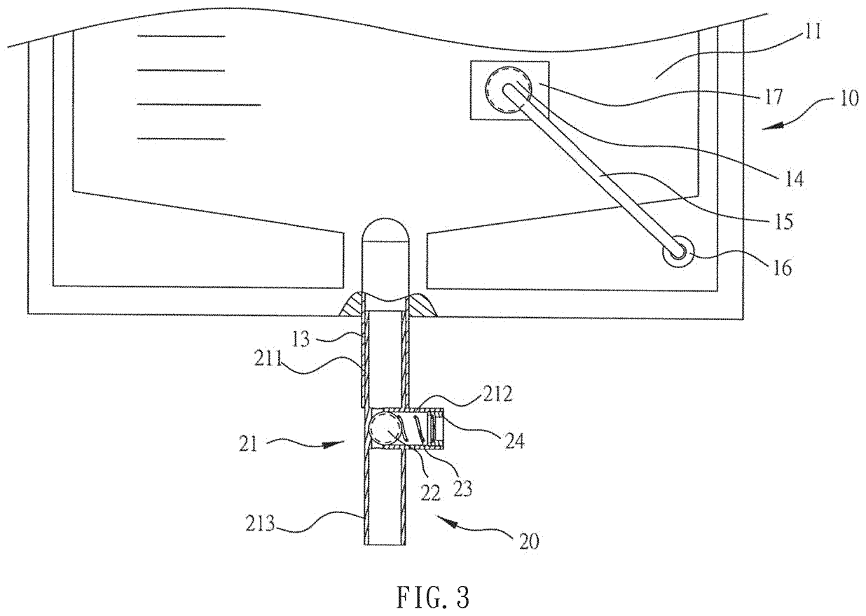

Referring to FIG. 1 to FIG. 7, a hand contamination preventing drainage bag structure is disclosed, comprising a urine bag 10 and a hand contamination preventing structure 20.

The urine bag 10 has a bag body 11 formed of a transparent or translucent resin sheet, with an inlet tube 12 connected to the upper part of the bag body 11. When the urine bag 10 is used, the catheter placed in the urethra of the patient is connected to the front end of the inlet tube 12. When the patient is on the bed, the bag body 11 is suspended on the frame of the bed through a suspending appliance. Further, a discharge tube 13 is connected to the lower part of the bag body 11 with respect to the inlet tube 12. A magnetic member 14 is disposed on one side of the bag body 11 in adjacent to the discharge tube 13. The magnetic member 14 is fixed to the fix portion 16 on the bag body 11 through a rope body 15. Also, a placement member 17 is disposed on the bag body 11 in adjacent to the discharge tube 13. The placement member 17 is formed of a metal material for the magnetic member 14 to be attached thereon.

The hand contamination preventing structure 20 is disposed on the discharge tube 13 of the urine bag 10. The hand contamination preventing structure 20 comprises a T-shaped tubular main body 21, a bead 22 comprising a metal material, a coil-shaped elastic member 23, and a plug member 24. The main body 21 comprises a first end 211, a second end 212, and a third end 213 that are interconnected. Therein, the first end 211 is inserted into the discharge tube 13 fixed to the bag body 11. The second end 212 contains the bead 22, the elastic member 23, and the plug member 24 sequentially disposed inside. The bead 22 is pressed by the plug member 24 through the elastic member 23 in the internal through hole among the first end 211, the second end 212, and the third end 213 of the T-shaped tubular main body 21, so that the first end 211, the second end 212, and the third end 213 of the T-shaped tubular main body 21 form a sealed operation status, in which the bead 22 is pressed by the plug member 24 through the elastic member 23 in the internal through hole combination among the first end 211, the second end 212, and the third end 213 of the T-shaped tubular main body 21, so as to form a sealed status in the main body 21 and the urine bag 10. It can be understood from the aforementioned structure combination that its structure is simpler, the manufacturing cost is lower, the price is cheaper, and the usage flexibility is optimal, achieving both economic and practical benefits.

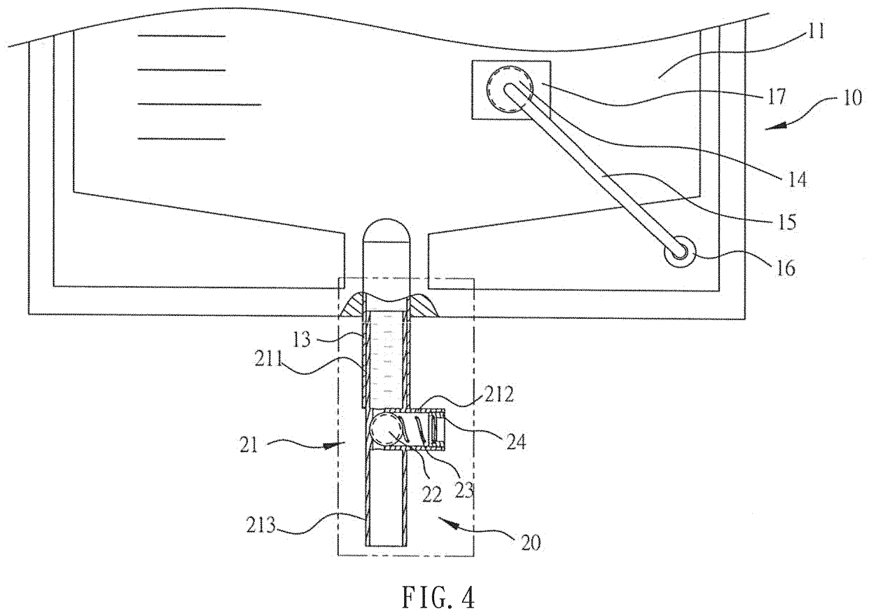

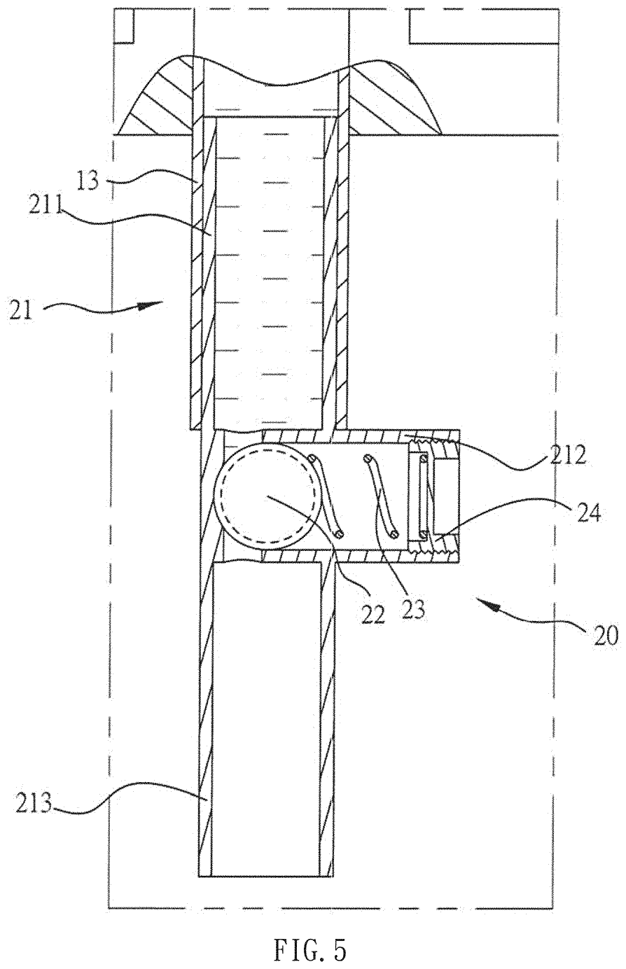

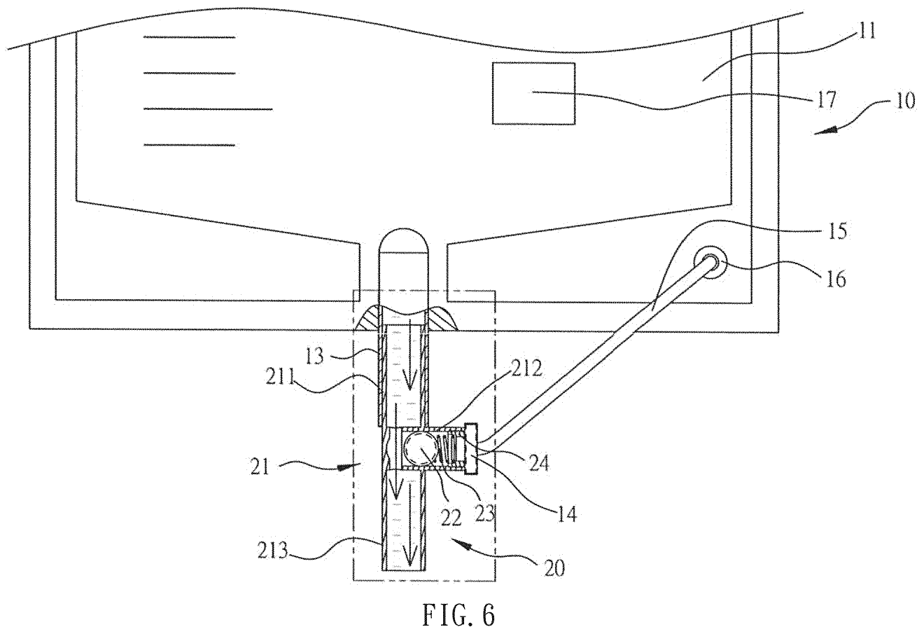

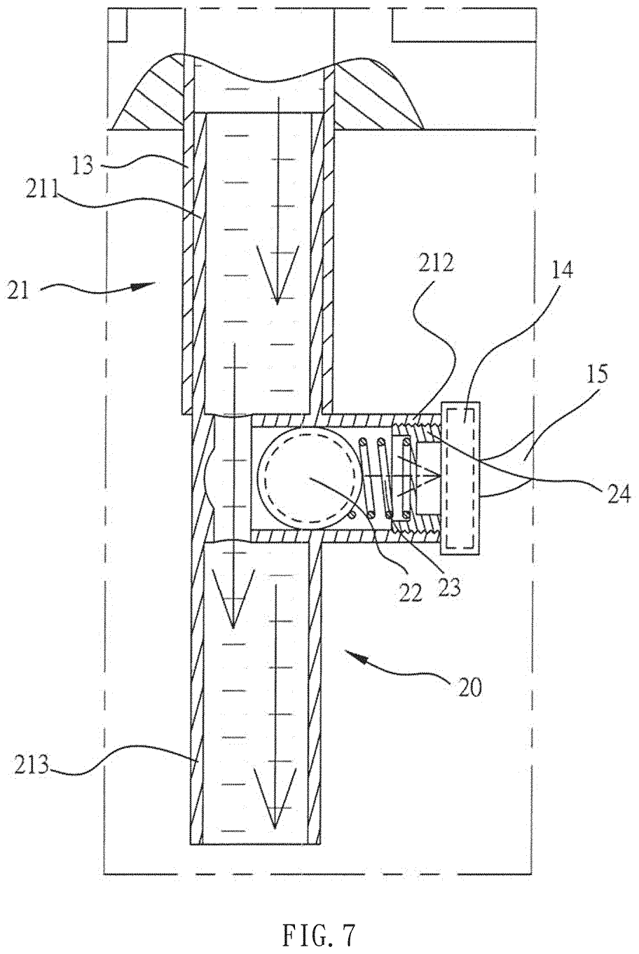

Referring to FIG. 4 to FIG. 7, when the present invention is being used. Referring to FIG. 4 and FIG. 5, the bead 22 is pressed by the plug member 24 through the elastic member 23 in the internal through hole combination among the first end 211, the second end 212, and the third end 213 of the T-shaped tubular main body 21, so as to form a sealed status in the main body 21 and the urine bag 10. Also referring to FIG. 6 and FIG. 7, when the present invention is to be used, the magnetic member 14 of the placement member 17 of the bag body 11 of the urine bag 10 is removed, and the magnetic member 14 is moved toward the second end 212 of the T-shaped tubular main body 21 in adjacent to the discharge tube 13, such that the bead 22 presses the elastic member 23, whereby the inner space between the first end 211 and the third end 213 of the T-shaped tubular main body 21 of the urine bag 10 is interconnected to form an open status. Therefore, medical staff can collect the urine discharge by the patient. Through such operation, the present invention achieves the convenience of labor-saving, simple, one-handed and fast operation, and also reduces the probability of the hand touching the urine. Thus, the improvement structure of the urine bag 10 has greater convenience of usage.

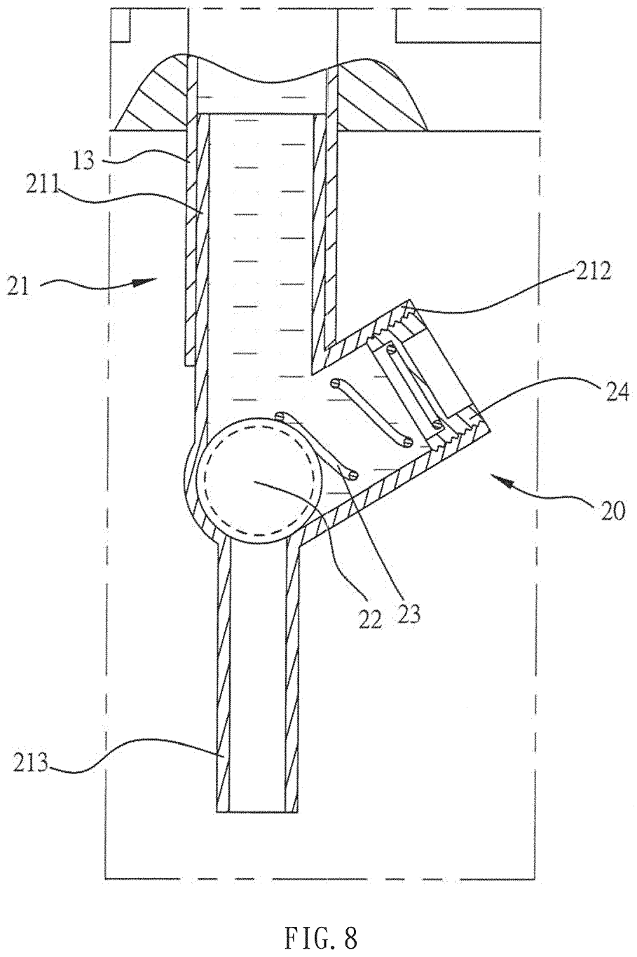

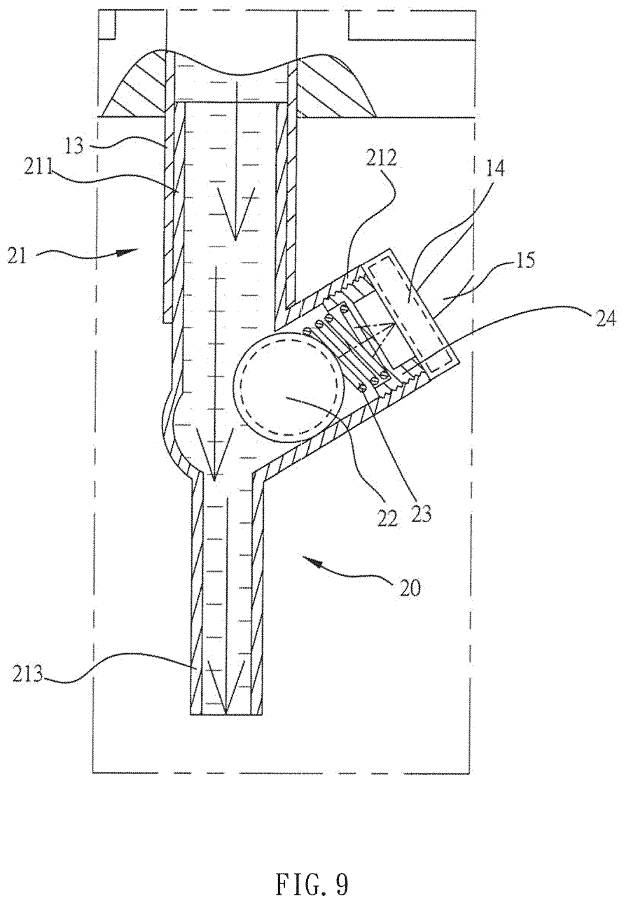

Referring to embodiments shown by FIG. 8 and FIG. 9, the hand contamination preventing structure 20 is disposed on the discharge tube 13 of the urine bag 10. The hand contamination preventing structure 20 comprises a Y-shaped tubular main body 21, a bead 22 comprising a metal material, a coil-shaped elastic member 23, and a plug member 24. The main body 21 comprises a first end 211, a second end 212, and a third end 213 that are interconnected. Therein, the first end 211 is inserted into the discharge tube 13 fixed to the bag body 11. The second end 212 contains the bead 22, the elastic member 23, and the plug member 24 sequentially disposed inside. The bead 22 is pressed by the plug member 24 through the elastic member 23 in the internal through hole among the first end 211, the second end 212, and the third end 213 of the Y-shaped tubular main body 21 , so that the first end 211, the second end 212, and the third end 213 of the Y-shaped tubular main body 21 form a sealed operation status, in which the bead 22 is pressed by the plug member 24 through the elastic member 23 in the internal through hole combination among the first end 211, the second end 212, and the third end 213 of the Y-shaped tubular main body 21, so as to form a sealed status in the main body 21 and the urine bag 10. Further, the bore diameter of the first end 211 and the second end 212 of the main body 21 is larger than the bore diameter of the third end 213.

Referring to FIG. 8 and FIG. 9, when the present invention is being used, the bead 22 is pressed by the plug member 24 through the elastic member 23 in the internal through hole combination among the first end 211, the second end 212, and the third end 213 of the Y-shaped tubular main body 21, so as to form a sealed status in the main body 21 and the urine bag 10. When the present invention is to be used, the magnetic member 14 of the placement member 17 of the bag body 11 of the urine bag 10 is removed, and the magnetic member 14 is moved toward the second end 212 of the Y-shaped tubular main body 21 in adjacent to the discharge tube 13, such that the bead 22 presses the elastic member 23, whereby the inner space between the first end 211 and the third end 213 of the Y-shaped tubular main body 21 of the urine bag 10 is interconnected to form an open status. Therefore, medical staff can collect the urine discharge by the patient. Through such operation, the present invention achieves the convenience of labor-saving, simple, one-handed and fast operation, and also reduces the probability of the hand. touching the urine. Thus, the improvement structure of the urine hag 10 has greater usage cleanliness.

With the foregoing configuration, effects of the present invention will be illustrated below.

In the hand contamination preventing drainage hag structure, the bead 22 is pressed by the plug member 24 through the elastic member 23 in the internal through hole combination among the first end 211, the second end 212, and the third end 213 of the T-shaped or Y-shaped tubular main body 21, so as to form a sealed status in the main body 21 and the urine bag 10. When the present invention is to be used, the magnetic member 14 of the placement member 17 of the bag body 11 of the urine bag 10 is removed, and the magnetic member 14 is moved toward the second end 212 of the T-shaped or Y-shaped tubular main body 21 in adjacent to the discharge tube 13, such that the bead 22 presses the elastic member 23, whereby the inner space between the first end 211 and the third end 213 of the T-shaped or Y-shaped tubular main body 21 of the urine bag 10 is interconnected to form an open status. Therefore, medical staff can collect the urine discharge by the patient. Through such operation, the present invention achieves the convenience of labor-saving, simple, one-handed and fast operation, and also reduces the probability of the hand touching the urine. Thus, the improvement structure of the urine bag 10 has greater convenience of usage.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A hand contamination preventing drainage bag structure, comprising:
a urine bag comprising a magnetic member; and
a hand contamination preventing structure disposed on the urine bag, the hand contamination preventing structure comprising a main body, a bead having a metal material, coil-shaped elastic member, and a plug member; the main body comprising a first end, a second end, and a third end that are interconnected; the first end being inserted into a discharge tube fixed to the bag body; the second end containing the bead, the elastic member, and the plug member sequentially disposed inside; the bead being pressed by the plug member through the elastic member in an internal through hole among the first end, the second end, and the third end of the main body, so that the first end, the second end, and the third end of the main body form a sealed operation status, in which the bead is pressed by the plug member through the elastic member in the internal through hole combination among the first end, the second end, and the third end of the main body, so as to form a sealed status in the main body and the urine bag; wherein
when the hand contamination preventing drainage bag structure is to be used, the magnetic member of the placement member of the bag body of the urine bag is removed, and the magnetic b is moved toward the second end of the main body in adjacent to the discharge tube, such that the bead presses the elastic member, whereby an inner space between the first end and the third end of the main body of the urine bag is inter to form an open status.

2. The hand contamination preventing drainage bag structure of claim 1, wherein the urine bag comprises a bag body, with an inlet tube connected to an upper part of the bag body, and a discharge tube connected to a lower part of the bag body with respect to the inlet tube; a magnetic member is disposed on one side of the bag body in adjacent to the discharge tube; the magnetic member is fixed to a fix portion on the bag body through a rope body; a placement member is disposed on the bag body in adjacent to the discharge tube; the placement member is formed of a metal material for the magnetic member to be attached thereon.

3. The hand contamination preventing drainage bag structure of claim 1, wherein the main body is formed as a T-shape tube.

4. A hand contamination preventing drainage bag structure, comprising:
a urine bag comprising a bag body, with an inlet tube connected to an upper part of the bag body; when the urine bag is being used, a catheter placed in a urethra of a patient is connected to a front end of the inlet tube; when the patient is on a bed, the bag body is suspended on the frame of the bed through a suspending appliance; a discharge tube is connected to a lower part of the bag body with respect to the inlet tube; a magnetic member is disposed on one side of the bag body in adjacent to the discharge tube, and the magnetic member is fixed to a fix portion on the bag body through a rope body; and
a hand contamination preventing structure disposed on the discharge tube of the urine bag, the hand contamination preventing structure comprising a Y-shaped tubular main body, a bead having a metal material, a cod-shaped elastic member, and a plug member; the main body comprising a first end, a second end, and a third end that are interconnected; the first end being inserted into a discharge tube fixed to the bag body; the second end containing the bead, the elastic member, and the plug member sequentially disposed inside; the bead being pressed by the plug member through the elastic member in an internal through hole among the first end, the second end, and the third end of the Y-shaped tubular main body, so that the first end, the second end, and the third end of the Y-shaped tubular main body form a sealed operation status, in which the bead is pressed by the plug member through the elastic member in the internal through hole combination among the first end, the second end, and the third end, so as to form a sealed status in the main body and the urine bag; wherein
when the hand contamination preventing drainage bag structure is to be used, the magnetic member of the placement member of the bag body of the urine bag is removed, and the magnetic member is moved toward the second end of the main body in adjacent to the discharge tube, such that the bead presses the elastic member, whereby an inner space between the first end and the third end of the main body of the urine bag is interconnected to form an open status.

5. The hand contamination preventing drainage bag structure of claim 4, wherein a bore diameter of the first end and the second end of the Y-shaped tubular main body is larger than a bore diameter of the third end.

6. The hand contamination preventing drainage bag structure of claim 4, wherein the bag body of the urine bag is formed of a transparent or translucent resin sheet.

7. The hand contamination preventing drainage bag structure of claim 4, wherein a placement member is disposed on the bag body in adjacent to the discharge tube; the placement member is formed of a metal material for the magnetic member to be attached thereon.

8. A hand contamination preventing drainage bag structure, comprising:
a urine bag comprising a magnetic member; and
a hand contamination preventing structure disposed on the urine bag, the hand contamination preventing structure comprising a main body, a bead having a metal material, an elastic member, and a plug member; the main body being interconnected and comprising the bead, the elastic member, and the plug member disposed inside; the bead being pressed by the plug member through the elastic member in an internal through hole in the main body, so as to form a sealed operation status, in which the bead is pressed by the plug member through the elastic member in the internal through hole combination, forming a sealed status in the main body and the urine bag; wherein
when the hand contamination preventing drainage bag structure is to be used, the magnetic member of the placement member of the bag body of the urine bag is removed, and the magnetic member is moved toward the main body, such that the bead presses the elastic member, whereby an inner space between the first end and the third end of the main body of the urine bag is interconnected to form an open status.
